(19) United States
(12) Patent Application Publication
(10) Pub. No.: US 2015/0157219 A1
(43) Pub. Date: Jun. 11, 2015

(54) BIOIMPEDANCE SENSOR ARRAY FOR HEART RATE DETECTION

(71) Applicant: Samsung Electronics Co., Ltd., Gyeonggi-do (KR)

(72) Inventors: Seulki Lee, Eindhoven (NL); Lindsay Brown, Mierlo (NL); Eva C. Wentink, Eindhoven (NL)

(73) Assignee: Samsung Electronics Co., Ltd., Gyeonggi-do (KR)

(21) Appl. No.: 14/282,950

(22) Filed: May 20, 2014

Related U.S. Application Data
(63) Continuation-in-part of application No. 14/103,717, filed on Dec. 11, 2013.
(60) Provisional application No. 61/969,763, filed on Mar. 24, 2014.

(51) Int. Cl. A61B 5/0205; A61B 5/0402; A61B 5/00

(52) U.S. Cl. CPC A61B 5/02055; A61B 5/681; A61B 5/742; A61B 5/7475; A61B 5/0402; A61B 5/0245

(57) ABSTRACT

Exemplary embodiments provide a bioimpedance sensor array for use in fluid flow detection applications, such as heart rate detection. Aspects of the exemplary embodiment include determining an optimal sub-array in a bioimpedance sensor array comprising more than four bioimpedance sensors arranged on a base such that the sensor array straddles or otherwise addresses a blood vessel when worn by a user; passing an electrical signal through at least a first portion of the bioimpedance sensors in the optimal sub-array to the user; measuring one or more bioimpedance values from the electrical signal using a second portion of the bioimpedance sensors in the optimal sub-array; and analyzing at least a fluid bioimpedance contribution from the one or more bioimpedance values.

### CROSS-REFERENCE TO RELATED APPLICATIONS

This application is a Continuation-In-Part of patent application Ser. No. 14/103,717 entitled “Self-Aligning Sensor Array” (Docket No. SSIC010US), filed on Dec. 11, 2013. This application further claims priority under 35 U.S.C. §120 of Patent Application Ser. No. 61/969,763, entitled “Bioimpedance Square Array Configuration for Heart Rate Detection” filed on Mar. 24, 2014, the contents of both applications are herein incorporated by reference.

### BACKGROUND

Heart rate may be measured, for example, by detecting the impedance changes caused by a pulse in blood flow within a local area of the body. A local measurement of heart rate is typically carried out, for example, on the chest, but other portions of the body containing arteries may also be used for a heart rate measurement, such as on the wrist.

Heart rate detection through measurement of the electrical properties of flowing blood may be achieved by measuring the potential created by current passed through the blood, artery, and surrounding tissue. In an alternating current measurement, the measured potential will be proportional to the current passed and the impedance of the area through which the current passes. Electrodes are used to carry out such a measurement. The electrodes are typically arranged in a two-wire arrangement in which the current is passed and the voltage measured between the same pair of electrodes. A problem with the two-wire arrangement is the introduction of contact (or lead) resistance which contributes an additive resistance term to the potential measurement (i.e. for a direct current measurement Ohm's law gives V=I*R where, in this case, R=resistance of sample+resistance of the contacts) and may be a substantial portion of the total measured resistance and thus may obscure measurement results, especially in low resistance samples.

A four-wire measurement may also be used that overcomes the contact resistance problem by passing current between two dedicated current electrodes and measuring potential between two dedicated voltage electrodes, all of which are arranged in an in-line configuration (the current electrodes being placed outside the voltage electrodes). In the four-wire electrode configuration, the voltage difference between current electrodes is separated out from the voltage measurement itself, thus minimizing their extraneous contribution.

In addition to in-line arrangements, current and voltage electrodes may be configured in a square layout. For thin-film impedance measurements, four electrodes may outline the shape of a square or rectangle (i.e., each electrode occupies a corner). This arrangement is used in the Van der Pauw method of measuring resistivity (or sheet resistance when substantially two-dimensional geometries are involved). In one implementation, two current and two voltage electrodes may be placed at the corners of a square outline and the current may flow along a single edge of the outlined square. The voltage may then be measured along the edge opposite to that of the current and the resistance between the current and voltage edges calculated using Ohm's law.

In-line four-wire bioimpedance measurement on an anterior side of a user's forearm, for example, the heart rate may be detected using bioelectrical impedance by placing four electrodes, two voltage electrodes flanked by two current electrodes, in a line along the radial artery.

However, in conventional implementations with electrode placement along the forearm, each of the electrodes are approximately 0.7 cm² or larger causing the full in-line arrangement of electrodes to require up to approximately 8 cm of space on the forearm. For many applications the space required by such an electrode arrangement is too great, for example, such large space requirements would limit the types and shapes of devices upon which an impedance-based heart rate detector may be mounted. If, for example, it is desired to place a heart rate detector within a watch-type host device, a more compact electrode arrangement would be required.

Accordingly, what is required is a bioimpedance measurement device usable in fluid flow detection applications, such as heart rate detection, and bioimpedance methods and host devices using such impedance measurement devices which utilize a compact electrode configuration while maintaining adequate measurement sensitivity.

### BRIEF SUMMARY

Exemplary embodiments provide a bioimpedance sensor array for use in fluid flow detection applications, such as heart rate detection. Aspects of the exemplary embodiment include determining an optimal sub-array in a bioimpedance sensor array comprising more than four bioimpedance sensors on a base such that the sensor array straddles or otherwise addresses a blood vessel when worn by a user; passing an electrical signal through at least a first portion of the bioimpedance sensors in the optimal sub-array to the user; measuring one or more bioimpedance values from the electrical signal using a second portion of the bioimpedance sensors in the optimal sub-array; and analyzing at least a fluid bioimpedance contribution from the one or more bioimpedance values.

According to the method and system disclosed herein, the exemplary embodiments provide an impedance measurement device that may be used in wearable devices that do not need exact placement above a wearer's blood vessel.

### BRIEF DESCRIPTION OF SEVERAL VIEWS OF THE DRAWINGS

These and/or other features and utilities of the present general inventive concept will become apparent and more readily appreciated from the following description of the embodiments, taken in conjunction with the accompanying drawings of which:

FIGS. 1A and 1B are diagrams illustrating embodiments of a modular wearable sensor platform.

FIG. 2 is a diagram illustrating one embodiment of a modular wearable sensor platform and components comprising the base module.

FIG. 3 is a block diagram illustrating an exemplary embodiment of a sensor array system for use in a wearable device, such as the modular wearable sensor platform.

FIG. 4 is a flow diagram illustrating a method of providing a bioimpedance sensor array and a method for using the bioimpedance sensor array to monitor and analyze physiological parameters, such as fluid flow, for applications including heart rate detection.

FIG. 5 is block diagram showing an exemplary bioimpedance sensor array.

FIGS. 6A through 6D are diagrams illustrating possible configurations of the current sensors and the voltage sensors in a 2×2 sub-array.

FIG. 6E shows a diagonal sub-array configuration of current sensors and voltage sensors that may be used in a 2×3 bioimpedance sensor

### DETAILED DESCRIPTION

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures.

Advantages and features of the present invention and methods of accomplishing the same may be understood more readily by reference to the following detailed description of embodiments and the accompanying drawings. The present general inventive concept may, however, be embodied in many different forms and should not be construed as being limited to the embodiments set forth herein. Rather, these embodiments are provided so that this disclosure will be thorough and complete and will fully convey the concept of the general inventive concept to those skilled in the art, and the present general inventive concept will only be defined by the appended claims. In the drawings, the thickness of layers and regions are exaggerated for clarity.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

The term “component” or “module”, as used herein, means, but is not limited to, a software or hardware component, such as a field programmable gate array (FPGA) or an application specific integrated circuit (ASIC), which performs certain tasks. A component or module may advantageously be configured to reside in the addressable storage medium and configured to execute on one or more processors. Thus, a component or module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for the components and components or modules may be combined into fewer components and components or modules or further separated into additional components and components or modules.

Unless defined otherwise, all technical and scientific terms used herein have the same meaning as commonly understood by one of ordinary skill in the art to which this invention belongs. It is noted that the use of any and all examples, or exemplary terms provided herein is intended merely to better illuminate the invention and is not a limitation on the scope of the invention unless otherwise specified. Further, unless defined otherwise, all terms defined in generally used dictionaries may not be overly interpreted.

Exemplary embodiments provide a bioimpedance measurement device usable in fluid flow detection applications, such as heart rate detection, and bioimpedance methods and host devices using such bioimpedance measurement devices are described. The bioimpedance sensor array may be configured as an X-by-Y array of more than four, and preferably at least six or eight, discrete bioimpedance sensors, including but not limited to electrodes. In one embodiment, at least one pair of electrodes in bioimpedance sensor array are determined as current electrodes that pass a sensing current and at least one other pair of electrodes are selected as voltage electrodes that measure potential difference or voltage. In one embodiment, the selection or determination of these pairs of current electrodes and voltage electrodes are fixed. In another embodiment, the selection or determination of the pairs of current electrodes and voltage electrodes is dynamic, such that the bioimpedance sensor array may be scanned to determine which selection of current and voltage electrodes provide an optimal signal quality.

The bioimpedance measurement device may be used in electronic devices employing bioimpedance measurement devices. Such electronic devices may include, but are not limited to, wearable devices and other portable and non-portable computing devices such as watches, cellular phones, smart phones, tablets, and laptops.

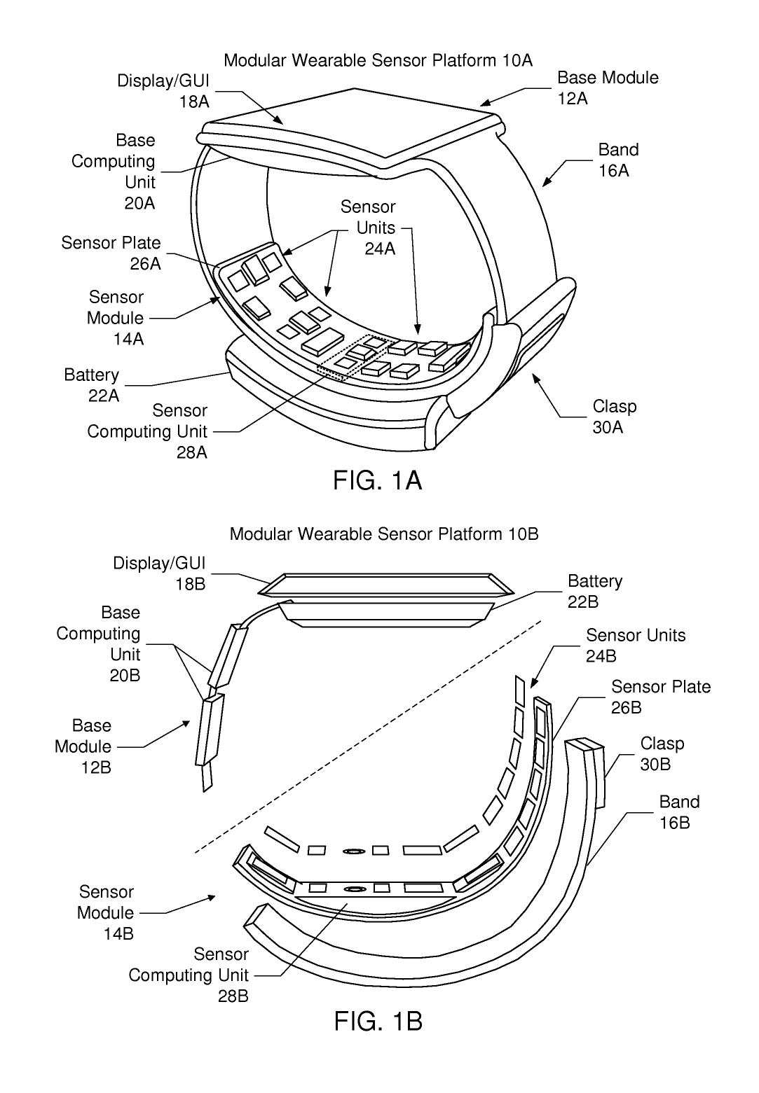

FIGS. 1A and 1B are diagrams illustrating embodiments of a modular wearable sensor platform. FIG. 1A depicts a perspective view of one embodiment of the wearable sensor platform 10A, while FIG. 1B depicts an exploded view of another embodiment of the wearable sensor platform 10B. Although the components of the wearable sensor platforms 10A and 10B (collectively wearable sensor platform 10) may be substantially the same, the locations of modules and/or components may differ. In the discussion of the specifics of FIGS. 1A and 1B, alphanumeric designations are used (e.g. 10A and 10B). However, to refer to either or both embodiments depicted in FIGS. 1A and 1B, numeric designations are used (e.g. 10 for 10A and/or 10B).

In the embodiment shown in FIG. 1A, the wearable sensor platform 10A may be implemented as a smart watch or other computing device that fits on a user's wrist. The wearable sensor platform 10A may include a base module 12A, a band 16A, a clasp 30A, a battery 22A and a sensor module 14A coupled to the band 16A. In some embodiments, the modules and/or components of the wearable sensor platform 10A may be removable by an end user. However, in other embodiments, the modules and/or components of the wearable sensor platform 10A are integrated into the wearable sensor platform 10A by the manufacturer and may not be intended to be removed by the end user.

The sensor module 14A may be positioned within the band 16A, such that the sensor module 14A is located at the bottom of the user's wrist in contact with the user's skin to collect physiological data from the user. The base module 12A attaches to the band 16A such that the base module 12A is positioned on top of the wrist.

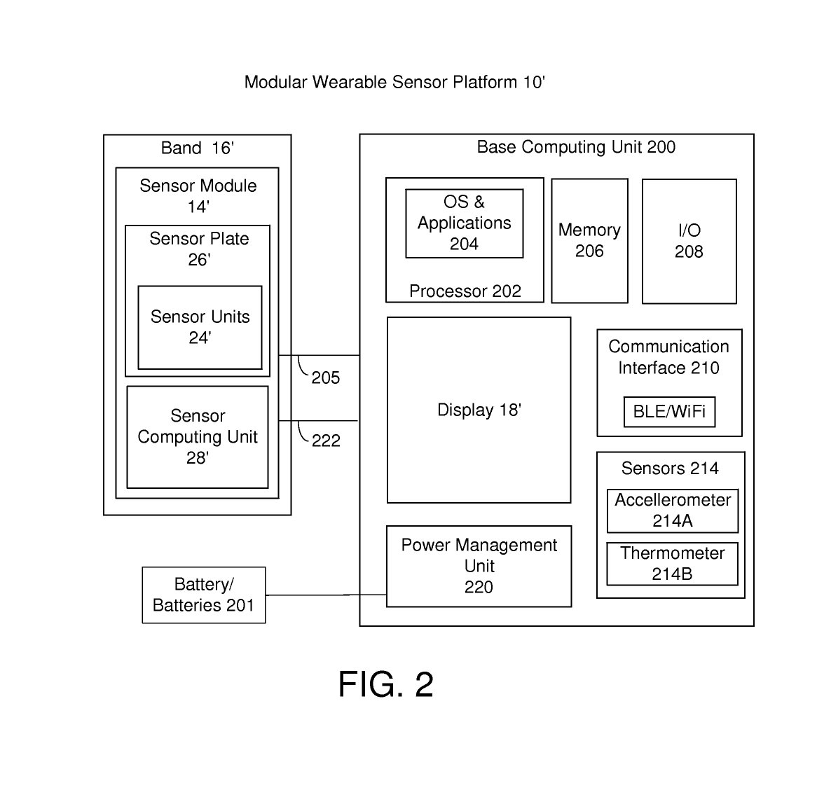

The base module 12A may include a base computing unit 20A and a display 18A on which a graphical user interface (GUI) may be provided. The base module 12A performs functions including but not limited to displaying time, performing calculations and/or displaying data including sensor data collected from the sensor module 14A. In addition to communication with the sensor module 14A, the base module 12A may wirelessly communicate with other sensor module(s) (not shown) worn on different body parts of the user to form a body area network. As will be discussed more fully with respect to FIG. 2, the base computing unit 20A may include a processor, memory, a communication interface and a set of sensors, such as an accelerometer and thermometer.

The sensor module 14A collects physiological data, activity data, sleep statistics and/or other data from a user and is in communication with the base module 12A. The sensor module 14A includes sensor units 24 housed in a sensor plate 26A. The sensor units 24A may include an optical sensor array, a thermometer, a galvanic skin response (GSR) sensor array, a bioimpedance (BioZ) sensor array, an electrocardiography sensor (ECG) sensor, or any combination thereof. Other sensor(s) may also be employed.

The sensor module 14A may also include a sensor computing unit 28A. The sensor computing unit 28A may analyze, perform calculations on and, in some embodiments, store the data collected by the sensor units 24A. The data from the sensor units 24A may also be provided to the base computing unit 20A for further processing. Because the sensor computing unit 28A may be integrated into the sensor plate 26A, it is shown by dashed lines in FIG. 1A. In other embodiments, the sensor computing unit 28A may be omitted. In such an embodiment, the base computing unit 20A may perform functions that would otherwise be performed by the sensor computing unit 28A. Through the combination of the sensor module 14A and base module 12A, data may be collected, stored, analyzed and presented to a user.

The wearable sensor platform 10B depicted in FIG. 1B is analogous to the wearable sensor platform 10A depicted in FIG. 1A. Thus, the wearable sensor platform 10B includes a band 16B, a battery 22B, a clasp 30B, a base module 12B including a display/GUI 18B and base computing unit 20B, and a sensor module 14B including sensor units 24B, a sensor plate 26B, and optional sensor computing unit 28B, which are analogous to the band 16A, the battery 22A, the clasp 30A, the base module 12A including the display/GUI 18A and base computing unit 20A and the sensor module 14A including sensor units 24A, the sensor plate 26A, and the optional sensor computing unit 28A, respectively. However, as can be seen in FIG. 1B, the locations of certain modules have been altered. For example, the clasp 30B is closer to the display/GUI 18B than the clasp 30A. Similarly, the battery 22B is housed with the base module 12B. In the embodiment shown in FIG. 1A, the battery 22A is housed with the band 16A, opposite to the display 18A. Thus, in various embodiments, the locations and/or functions of the modules may be changed.

In both embodiments shown in FIGS. 1A and 1B, the band or strap 16 may be one piece or modular. The band 16 may be made of a fabric. For example, a wide range of twistable and expandable elastic mesh/textiles are contemplated. The band 16 may also be configured as a multi-band or in modular links. The band 16 may include a latch or a clasp mechanism to retain the band on the user in certain implementations. In certain embodiments, the band 16 will contain wiring (not shown) connecting, among other things, the base module 12 and sensor module 14. Wireless communication, alone or in combination with wiring, between base module 12 and sensor module 14 is also contemplated.

FIG. 2 is a diagram illustrating one embodiment of a modular wearable sensor platform 10′ and components comprising the base module. The wearable sensor platform 10′ is analogous to the wearable sensor platforms 10 and thus includes analogous components having similar labels. In this embodiment, the wearable sensor platform 10′ may include a band 16′, and a sensor module 14′ attached to band 16′. The removable sensor module 14′ may further include a sensor plate 26′ attached to the band 16′, and sensor units 24′ attached to the sensor plate 26′. The sensor module 14′ may also include a sensor computing unit 28′.

The wearable sensor platform 10′ includes a base computing unit 200 analogous to the base computing unit 20 and one or more batteries 201. For example, permanent and/or a removable batteries that are analogous to the battery 22 may be provided. In one embodiment, the base computing unit 200 may communicate with the sensor computing unit 28′ through a communication interface 205. In one embodiment, the communications interface 205 may comprise a serial interface. The base computing unit 200 may include a processor 202, a memory 206, input/output (I/O) 208, a display 18′, a communication interface 210, sensors 214, and a power management unit 220.

The processor 202, the memory 206, the I/O 208, the communication interface 210 and the sensors 214 may be coupled together via a system bus (not shown). The processor 202 may include a single processor having one or more cores, or multiple processors having one or more cores. The processor 202 may execute an operating system (OS) and various applications 204. Examples of the OS may include, but not limited to, Linux and Android™.

According to the exemplary embodiment, the processor 202 may execute a calibration and data acquisition component (not shown) that may perform sensor calibration and data acquisition functions. In one embodiment, the sensor calibration function may comprise a process for self-aligning one more sensor arrays to a blood vessel. In one embodiment, the sensor calibration may be performed at startup, prior to receiving data from the sensors, or at periodic intervals during operation.

The memory 206 may comprise one or more memories comprising different memory types, including DRAM, SRAM, ROM, cache, virtual memory and flash memory, for example. The I/O 208 may comprise a collection of components that input information and output information. Example components comprising the I/O 208 include a microphone and speaker.

The communication interface 210 may include a wireless network interface controller (or similar component) for wireless communication over a network. In one embodiment, example types of wireless communication may include Bluetooth Low Energy (BLE) and WLAN (wireless local area network). However, in another embodiment, example types of wireless communication may include a WAN (Wide Area Network) interface, or a cellular network such as 3G, 4G or LTE (Long Term Evolution).

In one embodiment, the display 18′ may be integrated with the base computing unit 200, while in another embodiment, the display 18′ may be external from the base computing unit 200. The sensors 214 may include any type of microelectromechanical systems (MEMs) sensor, such as an accelerometer/gyroscope 214A and a thermometer 214B, for instance.

The power management unit 220 may be coupled to the battery/batteries 201 and may comprise a microcontroller that governs power functions of the base computing unit 200. In one embodiment, the power management unit 220 may also control the supply of battery power to the removable sensor module 14′ via power interface 222.

Although not shown, the base computing unit 200 may optionally include an electrocardiography sensors (ECG) and bioimpedance (BIOZ) analog front end (AFE), a galvanic skin response (GSR) AFE, and an optical sensor AFE, depending on the type of sensor units 24 equipped on the sensor module 14.

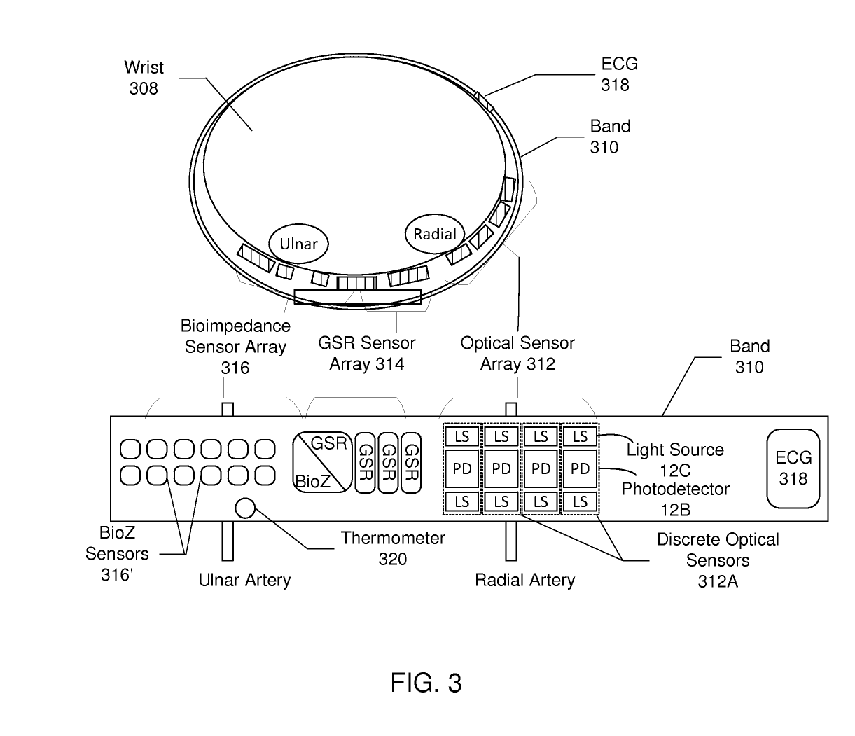

FIG. 3 is a block diagram illustrating an exemplary embodiment of a sensor array system for use in a wearable device, such as the modular wearable sensor platform. The system includes a band 310 that may house one or more self-aligning sensors arrays. In one embodiment, the band 310 corresponds to band 16 of the modular wearable sensor platform 10, with or without use of the sensor plate 26. In another embodiment, the band 310 may be a single device that is not part of the modular wearable sensor platform 10.

The top portion of FIG. 3 shows the band 310 wrapped around a cross-section of a user's wrist 308, while the bottom portion of FIG. 3 shows the band 310 in an unrolled position. According to one embodiment, the band 310 includes a bioimpedance (BioZ) sensor array 316, and optionally, an optical sensor array 312, a galvanic skin response (GSR) sensor array 314, an electrocardiography sensor (ECG) 318, or any combination thereof.

According to one exemplary embodiment, the sensor arrays 316, 314 and 312 each comprise an array of discrete sensors that are arranged or laid out on the band 310, such that when the band 310 is worn on a body part, each sensor array straddles or otherwise addresses a particular blood vessel (i.e., a vein, artery, or capillary), or an area with higher electrical response irrespective of the blood vessel. More particularly, each of the sensor arrays 316, 314, and 312 may be laid out substantially perpendicular to a longitudinal axis of the blood vessel and overlaps a width of the blood vessel to obtain an optimum signal. In one embodiment, the band 310 may be worn so that the self-aligning sensor arrays 316, 314, and 312 on the band 310 contact the user's skin, but not so tightly that the band 310 is prevented from any movement over the body part, such as the user's wrist 308.

As used herein, the bioimpedance (BioZ) sensor array 316 comprises an impedance measurement device usable in fluid flow detection applications, such as heart rate detection, of a living biological subject. The BioZ sensor array 316 and bioimpedance methods may be used in conjunction with a host electronic devices, including but not limited to, the base computing unit 200. Other examples of host electronic devices include, but are not limited to, other types of wearable devices and portable and non-portable computing devices such as cellular phones, smart phones, tablets, and laptops.

Conventional bioimpedance sensors typically comprise a single pair of electrodes, one electrode for the “I” current and the other electrode for the “V” voltage that measure bioelectrical impedance or opposition to a flow of electric current through the tissue.

However, according to one embodiment, the bioimpedance sensor array 316 is provided comprising more than four bioimpedance sensors 316′ and that straddles a blood vessel of a user when worn. In one embodiment, any pair of bioimpedance sensors 360′ may be selected to form a current pair “I” and another pair may be selected to form a voltage pair “V”, and as explained below. In one embodiment, the selection is fixed. In another embodiment, the selection is dynamic and performed during operation of the bioimpedance sensor array 316. The dynamic selection could be made using a multiplexor (not shown). In the embodiment shown, the bioimpedance sensor array 316 is shown straddling an artery, such as the radial or ulnar artery. In one embodiment, one or more of the BioZ sensors 316′ may be multiplexed with one or more of the GSR sensors 314.

In one embodiment, the optical sensor array 312 may comprise a photoplethysmograph (PPG) sensor array that may measures relative blood flow, pulse and/or blood oxygen level. In this embodiment, the optical sensor array 312 may be arranged on the band 310 so that the optical sensor array 312 straddles or otherwise addresses an artery, such as the radial or ulnar artery. In one embodiment, the optical sensor array 312 may include an array of discrete optical sensors 312A, where each discrete optical sensor 312A is a combination of at least one photodetector 12B and at least two matching light sources 312C located adjacent to the photodetector 312B. In one embodiment, each of the discrete optical sensors 312A may be separated from its neighbor on the band 310 by a predetermined distance of approximately 0.5 to 2 mm.

In one embodiment, the light sources 12C may each comprise light emitting diode (LED), where LEDs in each of the discrete optical sensors 312A emit a light of a different wavelength. Example light colors emitted by the LEDs may include green, red, near infrared, and infrared wavelengths. Each of the photodetectors 312B convert received light energy into an electrical signal. In one embodiment, the signals may comprise reflective photoplethysmograph signals. In another embodiment, the signals may comprise transmittance photoplethysmograph signals. In one embodiment, the photodetectors 312B may comprise phototransistors. In alternative embodiment, the photodetectors 312B may comprise charge-coupled devices (CCD).

The galvanic skin response (GSR) sensor array 314 may comprise four or more GSR sensors that may measure electrical conductance of the skin that varies with moisture level. Conventionally, two GSR sensors are necessary to measure resistance along the skin surface. According to one aspect of one embodiment, the GSR sensor array 314 is shown including four GSR sensors, where any two of the four may be selected for use. In one embodiment, the GSR sensors 314 may be spaced on the band 2 to 5 mm apart.

In yet another embodiment, the band 310 may include one or more electrocardiography sensors (ECG) 318 (one on the inside of the band facing the skin and another on the outside of the band) that measure electrical activity of the user's heart over a period of time. In addition, the band 310 may also include a thermometer 320 for measuring temperature or a temperature gradient.

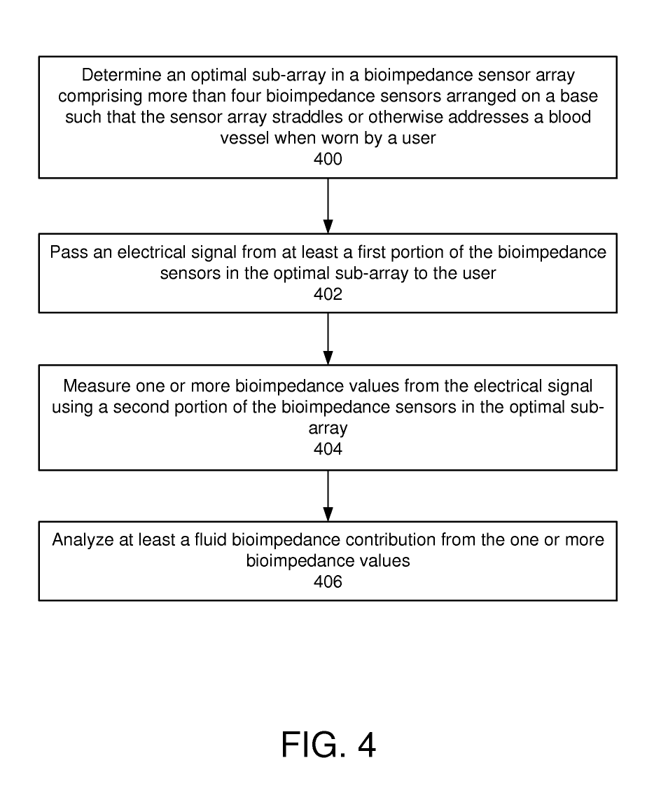

FIG. 4 is a flow diagram illustrating a method of providing a bioimpedance sensor array and a method for using the bioimpedance sensor array to monitor and analyze physiological parameters, such as fluid flow, for applications including heart rate detection. In one embodiment the process may be performed by one or more software components (e.g., a calibration and data acquisition component) executing on a processor coupled to the sensor array. The processor may correspond to the sensor computing unit 28, the processor 202 of the base computing unit 200 (shown in FIG. 2), and/or a separate processor.

According to the exemplary embodiment, the process may begin by determining an optimal sub-array in a bioimpedance sensor array comprising more than four bioimpedance sensors arranged on a base, such that the bioimpedance sensor array straddles or otherwise addresses a blood vessel when worn by a user (block 400). In one embodiment, the optimal sub-array may comprise any pair of bioimpedance sensors selected to form a current pair “I” and another pair selected to form a voltage pair “V”.

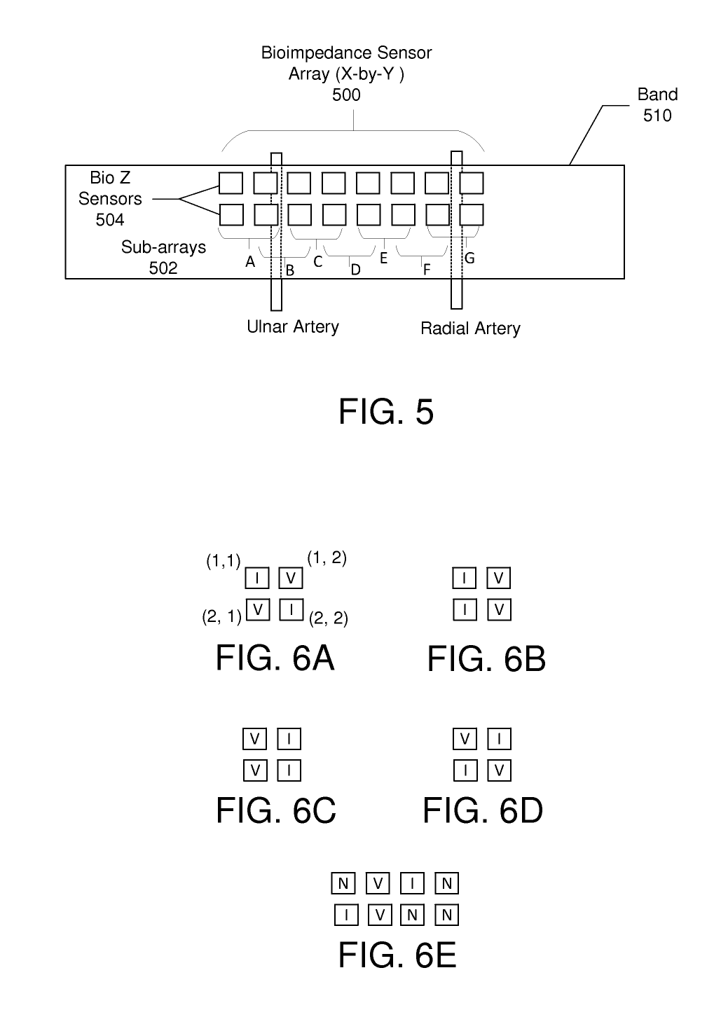

FIG. 5 is block diagram showing an exemplary bioimpedance sensor array. According to one embodiment, the bioimpedance sensor array 500 may be configured as an X-by-Y array of more than four, and preferably at least six or eight, discrete bioimpedance sensors 504. The X-by-Y bioimpedance sensor array 500 may be placed over any appropriate measurement site. Using heart rate measurement as an example, the sensors may be placed upon the underside of a wearer's forearm (i.e. the palm side) or another body part. The position of the sensor array upon the underside of the forearm may further be refined to a position above an artery, such as the radial or ulnar arteries where the sensor positioning relative to the arteries may be such that either artery may be located anywhere within the area defined by the bioimpedance sensor array 500 as long as the blood pulse travels between the pairs of current and voltage sensors. In the embodiment shown, the bioimpedance sensor array 500 is shown positioned over both the ulnar artery and the radial artery. However, in another embodiment, the bioimpedance sensor array 500 may be placed over only one of the arteries or over other blood vessels.

According to one aspect of the exemplary embodiment, at least one M-by-N sub-array 502A through 502G (collectively sub-arrays 502) of the X-by-Y bioimpedance sensor array 500 is selected as the optimal sub-array. In this embodiment, the optimal sub-array of bioimpedance sensors refers to a particular set of discrete bioimpedance sensors 504 having an optimum position over the blood vessel and therefore provide optimal signal quality.

In one embodiment, at least one pair of the bioimpedance sensors in the optimal sub-array 502 is selected as current sensors, and at least one other pair is selected as voltage sensors. Beyond that, additional bioimpedance sensors 504 in the bioimpedance sensor array 500 may be selected as either current or voltage sensors or unused. In one embodiment selection of the current sensors and the voltage sensors does not necessarily require selection of bioimpedance sensors in adjacent rows or columns of the bioimpedance sensor array.

As shown in FIG. 5, one possible configuration of the M-by-N sub-arrays 502 may comprise a 2×2 square sensor arrangement. In one embodiment, adjacent M-by-N sub-arrays 502 are electrically joined together to form the full X-by-Y bioimpedance sensor 500. In the example shown, four 2-by-2 sub-arrays 502 are shown placed in a row adjacent to one another to form the single 2-by-8 bioimpedance sensor array 500.

In one embodiment, configuration and placement of the sub-arrays 502 is fixed, where each of the sub-arrays 502 includes at least two current sensors and at least two voltage sensors. For example, sub-arrays A, C, E and G may be fixed, and during operation, one of these sub-arrays is selected as the optimal sub-array.

In another embodiment, configuration of the sub-arrays 502 is dynamic. In this embodiment, during calibration, the bioimpedance sensor array 500 is scanned to identify which sets of bioimpedance sensors provide the optimal signal and using the identified sets of bioimpedance sensors as the optimal sub-array. In one embodiment, during this process the discrete bioimpedance sensors 504 may be activated in series. In an alternative embodiment, the discrete bioimpedance sensors 504 may be activated in parallel. Thereafter, a first portion of the bioimpedance sensors in the optimal sub-array that provide an optimum signal are selected as current sensors, and a second portion of the bioimpedance sensors in the optimal sub-array are selected as voltage sensors. For example, in FIG. 5, any of the sub-arrays 502 A through G could be determined to be the optimal sub-array. Other sub-arrays are also possible but not illustrated. Also, after a predetermined time period, or at regular time intervals, the determination of the optimal sub-array may be performed again to see if a better setting exists to improve performance.

FIGS. 6A through 6D are diagrams illustrating possible configurations of the current sensors and the voltage sensors in a 2×2 sub-array. For explanation purposes, FIG. 6A shows that the illustrated example assumes that the 2×2 sub-array is in a row (x) and column (y) format with indices (1, 1), (1, 2), (2, 1) and (2, 2).

FIG. 6A shows a configuration of the current sensors “I” and the voltage sensors “V” in the 2×2 sub-array as: (1, 1)=I, (1, 2)=V, (2, 1)=V and (2, 2)=I.

FIG. 6B shows a configuration of the current sensors “I” and the voltage sensors “V” in the 2×2 sub-array as: (1, 1)=I, (1, 2)=V, (2, 1)=I and (2, 2)=V.

FIG. 6C shows a configuration of the current sensors “I” and the voltage sensors “V” in the 2×2 sub-array as: (1, 1)=V, (1, 2)=I, (2, 1)=V and (2, 2)=I.

FIG. 6D shows a configuration of the current sensors “I” and the voltage sensors “V” in the 2×2 sub-array as: (1, 1)=V, (1, 2)=I, (2, 1)=I and (2, 2)=V.

FIG. 6E shows a diagonal sub-array configuration of current sensors and voltage sensors that may be used in a 2×3 bioimpedance sensor, for example, where N represents unused sensors in the six sensor array. As shown, adjacent voltage and current sensors (“V”, “I”) in the first row of the array is offset by one column from adjacent current and voltage sensors in the second row of the array (“I”, “V”).

Using heart rate measurement on a wrist as an example, the optimal sub-array may be located above the radial or ulnar arteries, where sub-array positioning relative to the arteries may be such that either artery may be located anywhere within the area defined by the optimal sub-array as long as fluid, e.g., blood, pulses travel between pairs of current and voltage sensors. However, optimal sub-array placement relative to the radial and/or ulnar artery may not necessarily require that the radial and/or ulnar artery lie directly between two of the bioimpedance sensors 500 in the optimal sub-array. But as long as an outer perimeter of the optimal sub-array substantially overlays the radial and/or ulnar artery (or other blood vessel), a measurement adequate to deduce heart rate may still be obtained.

One skilled in the art may readily recognize that additional sensors may be used and/or arranged in a variety of array-type configurations to form different shapes and to effectively increase the sensing area covered by the sensor array, thus allowing greater robustness in placement of the sensor device as long as at least one of the sub-arrays overlays a blood vessel.

In one embodiment, each of the bioimpedance sensors 504 may comprise electrodes. The electrodes may be, for example, within a size range of approximately 0.1 to 1.0 cm² and separated, for example, by a distance of approximately 0.1 to 1.0 cm. The electrode size is proportional to required placement distance between electrodes, so smaller electrodes should be placed closer together. The electrodes may be constructed from a number of conductive materials. In one embodiment, the electrode material may comprise at least one of a metallic material including gold, stainless steel, nickel, and other metallic elements, compounds, or alloys. In another embodiment, the electrode material may comprise a coating on a non-conductive material such as, for example, a polymer or a ceramic coated with Ag/AgCl. However, additional conductor/non-conductor material combinations may be used (e.g. additional noble metal and metal-halide combinations). In another embodiment combinations of materials may be used including, for example, a conductive rubber with an Ag/AgCl coating.

Referring again to FIG. 4, the processor may be configured to pass an electrical signal through at least a first portion of the bioimpedance sensors in the optimal sub-array to the user (block 402).

In one embodiment, the electrical signal or signals may comprise a current that is passed between two current sensors. The electrical signal should preferably intersect the path of fluid flow to be measured. In an embodiment, the electrical signal may be modified, for example, by adjusting electrical signal parameters, including frequency, amplitude, waveform, or any combinations thereof, as necessary to provide an optimal measurement. In one embodiment, the electrical signal parameters may be changed in response to a quality of any sensed signals. According to one embodiment, the sensing method may further include making a series of measurements with different electrical signal parameters and sensed signals may be compared in order to select the best measurement.

Referring still to FIG. 4, one or more bioimpedance values are measured from the electrical signal using a second portion of the bioimpedance sensors in the optimal sub-array (block 404). In one embodiment, the bioimpedance values may be measured by sensing a potential or voltage between two voltage sensors/electrodes in the bioimpedance sensor array. In one embodiment, this sensing of the potential preferably intersects a path of fluid flow to be measured. In yet another embodiment, bioimpedance values from adjacent electrodes may be measured.

Finally, at least a fluid bioimpedance contribution is then measured from the one or more bioimpedance values (block 406). The fluid bioimpedance being measured may include various fluid types including, for example, flowing bodily fluids such as blood flowing through an artery.

A method and system for providing a bioimpedance sensor array for heart rate detection has been disclosed. The present invention has been described in accordance with the embodiments shown, and there could be variations to the embodiments, and any variations would be within the spirit and scope of the present invention. For example, one embodiment can be implemented using hardware, software, a computer readable medium containing program instructions, or a combination thereof. Software written according to the present invention is to be either stored in some form of computer-readable medium such as a memory, a hard disk, or a CD/DVD-ROM and is to be executed by a processor. Accordingly, many modifications may be made by one of ordinary skill in the art without departing from the spirit and scope of the appended claims.

We claim:

1. A method for providing a bioimpedance sensor array, comprising:
determining an optimal sub-array in a bioimpedance sensor array comprising more than four bioimpedance sensors arranged on a base such that the sensor array straddles or otherwise addresses a blood vessel when worn by a user;
passing an electrical signal through at least a first portion of the bioimpedance sensors in the optimal sub-array to the user;
measuring one or more bioimpedance values from the electrical signal using a second portion of the bioimpedance sensors in the optimal sub-array; and
analyzing at least a fluid bioimpedance contribution from the one or more bioimpedance values.

2. The method of claim 1, further comprising: selecting at least one pair of the bioimpedance sensors in the optimal sub-array to form current sensors and selecting at least one other pair to form voltage sensors.

3. The method of claim 1, wherein configuration and placement of the optimal sub-array is fixed.

4. The method of claim 1, wherein configuration and placement of the optimal sub-array is dynamic.

5. The method of claim 4, further comprising: scanning the bioimpedance sensor array to identify which sets of bioimpedance sensors provide an optimal current signal and using the identified sets of bioimpedance sensors as the optimal sub-array; selecting a first portion of the bioimpedance sensors in the optimal sub-array that provides an optimum current signal as current sensors; and selecting a second portion of the bioimpedance sensors in the optimal sub-array as voltage sensors.

6. The method of claim 5, wherein the optimal sub-array is positioned relative to the blood vessel such that the blood vessel is located anywhere within an area defined by the optimal sub-array as long as blood pulses travel between pairs of the current sensors and the voltage sensors.

7. The method of claim 1, further comprising: multiplexing one or more of the bioimpedance sensors with one or more galvanic skin response (GSR) sensors.

8. The method of claim 1, wherein the bioimpedance sensors comprise electrodes.

9. The method of claim 8, wherein a size of the electrodes size proportional to required placement distance between the electrodes, such that smaller electrodes are placed closer together.

10. The method of claim 8, wherein the electrodes are within a size range of approximately 0.1 to 1.0 cm² and separated by a distance of approximately 0.1 to 1.0 cm.

11. The method of claim 8, wherein the electrodes comprise at least one of a metallic material including gold, stainless steel, nickel, and other metallic elements, compounds, or alloys.

12. The method of claim 8, wherein the electrodes comprise a polymer or a ceramic coated with Ag/AgC.

13. The method of claim 8, wherein the electrodes comprise a conductive rubber with an Ag/AgCl coating.

14. The method of claim 1, wherein passing an electrical signal further comprises: modifying the electrical signal by adjusting signal parameters, including frequency, amplitude, waveform, or any combination thereof, to provide an optimal measurement.

15. The method of claim 14, further comprising: making a series of measurements using different signal parameters.

16. A bioimpedance sensor array, comprising:
an array of more than four bioimpedance sensors arranged on the base such that the sensor array straddles or otherwise addresses a blood vessel when worn by a user; and
a processor coupled to the sensor array configured to:
determine an optimal sub-array in the bioimpedance sensor array;
pass an electrical signal through at least a first portion of the bioimpedance sensors in the optimal sub-array to the user;
measure one or more bioimpedance values from the electrical signal using a second portion of the bioimpedance sensors in the optimal sub-array; and
analyze at least a fluid bioimpedance contribution from the one or more bioimpedance values.

17. The system of claim 16, further comprising: selecting at least one pair of the bioimpedance sensors in the optimal sub-array to form a current sensors and selecting at least one other pair to form voltage sensors.

18. The system of claim 16, wherein configuration and placement of the sub-race is fixed.

19. The system of claim 18, wherein configuration and placement of the sub-arrays is dynamic.

20. The system of claim 19, wherein the processor scans the bioimpedance sensor array to identify which sets of bioimpedance sensors provide an optimal current signal and uses the identified sets of bioimpedance sensors as the optimal sub-array; and selects a second portion of the bioimpedance sensors in the optimal sub-array as voltage sensors.

21. The system of claim 20, wherein the optimal sub-array is positioned relative to the blood vessel such that the blood vessel is located anywhere within an area defined by the optimal sub-array as long as blood pulses travel between pairs of the current sensors and the voltage sensors.

22. The system of claim 16, wherein one or more of the bioimpedance sensors are multiplexed with one or more galvanic skin response (GSR) sensors.

23. The system of claim 16, wherein the bioimpedance sensors comprise electrodes.

24. The system of claim 23, wherein a size of the electrodes size proportional to required placement distance between the electrodes, such that smaller electrodes are placed closer together.

25. The system of claim 23, wherein the electrodes are within a size range of approximately 0.1 to 1.0 cm² and separated by a distance of approximately 0.1 to 1.0 cm.

26. The system of claim 23, wherein the electrodes comprise at least one of a metallic material including gold, stainless steel, nickel, and other metallic elements, compounds, or alloys.

27. The system of claim 23, wherein the electrodes comprise a polymer or a ceramic coated with Ag/AgC.

28. The system of claim 23, wherein the electrodes comprise a conductive rubber with an Ag/AgCl coating.

29. The system of claim 16, wherein the electrical signal is modified by adjusting signal parameters, including frequency, amplitude, waveform, or any combination thereof, to provide an optimal measurement.

30. The system of claim 29, wherein a series of measurements is made using different signal parameters.
